(19) United States
(12) Patent Application Publication
(10) Pub. No.: US 2003/0186948 A1
(43) Pub. Date: Oct. 2, 2003

(54) O-linked N-acetylglucosamine pathway in the pathogenesis of neurodegeneration and diabetes

(76) Inventors: Jeffrey Kudlow, Birmingham, AL (US); Robert Konrad, Carmel, IN (US)

Correspondence Address: Benjamin Aaron Adler, ADLER & ASSOCIATES, 8011 Cahdle Lane, Houston, TX, 77071, US

(21) Appl. No.: 10/392,508

(22) Filed: Mar. 20, 2003

Related U.S. Application Data
(63) Continuation-in-part of application No. 09/813,534, filed on Mar. 21, 2001, now Pat. No. 6,589,995.
(60) Provisional application No. 60/190,785, filed on Mar. 21, 2000.

(51) Int. Cl. A61K 31/7008; A61K 31/655; A61K 31/519; A61K 31/4162

(52) U.S. Cl. 514/150; 514/262.1; 514/62; 514/389

(57) ABSTRACT

O-linked protein glycosylation of proteasome mediated by O-linked N-acetylglucosamine transferase (OGT) blocks proteasomal function. This blockade of proteasomal function results in accumulation of proapoptotic factors that lead to neuro-endocrine cell death in the pathogenesis of neurodegenerative diseases and diabetes. Thus, inhibiting OGT activity by structural analog of N-acetylglucosamine such as (Z)-1-[N-(3-Ammoniopropyl)-N-(n-propyl)amino]diazen-ium-1,2-diolate would provide new methods of preventing and/or treating late onset of Alzheimer's disease and diabetes.

### CROSS-REFERENCE TO RELATED APPLICATION

This is a continuation-in-part application of U.S. Ser. No. 09/813,534, filed Mar. 21, 2001, which claims the benefit of provisional patent application U.S. Serial No. 60/190,785, filed Mar. 21, 2000, now abandoned.

### FEDERAL FUNDING LEGEND

This invention was produced in part using funds obtained through a grant (NIH DK55262) from the National Institutes of Health. Consequently, the federal government has certain rights in this invention.

### BACKGROUND OF THE INVENTION

1. Field of the Invention

The present invention relates generally to the field of neuro-endocrine cell death. More specifically, the present invention relates to new methods of preventing and/or treating neurodegenerative diseases and diabetes by inhibiting O-linked protein glycosylation.

2. Description of the Related Art

Neurodegenerative Disorders and Proteasomes

Disease-specific protein aggregates are the hallmark of many of the neurodegenerative disorders. The most common of these disorders is Alzheimer's disease in which the aggregate is composed mainly of β-amyloid (Aβ) in the extracellular space and tau protein in the intracellular neurofibrillary tangles. Parkinson's disease, polyglutamine disease, familial amyotrophic lateral sclerosis and even prion disease (Jakob-Creutzfeld) are all characterized by their specific protein aggregates and inclusions.

For these aggregates to develop, either there must be a defect that increases the synthesis of the protein from which the aggregate is composed or a defect in the degradation of the protein. Indeed, there are examples of both. For Alzheimer's disease, mutations in the Aβ precursor protein (APP) or mutations in the presenilin 1 or 2 result in a greater rate of conversion of APP to Aβ by the secretases. It is the Aβ with its tendency to aggregrate that is thought to result in the initiation of the disease.

On the other hand, there are example of delayed clearance of the aggregating protein that also results in disease. For example, in Parkinison's disease, one of the mutations that is associated with this condition is in a gene that encodes a ubiquitin COOH-terminal hydrolase. It is thought that partial loss of catalytic activity of the mutant thiol protease leads to the accumulation of α-synuclein in the neurons of the substantia nigra. This aggregate accumulation and formation of the characteristic Lewy bodies is associated with toxicity to these neurons leading to their premature failure and early onset Parkinson's disease.

The idea that these aggregates accumulate because they are not disposed of rapidly enough has given rise to the general idea that a failure of the ubiquitin-proteasome system somehow underlies the pathogenesis of these conditions. The proteasome is the major cellular organelle that is involved in the destruction of intracellular proteins. It is present in both prokaryotes and eukaryotes. The structure of the eukaryotic proteasome has been elucidated by x-ray crystallography. It consists of a 20S subunit that forms a cylindrical structure. The cylinder consists of two copies each of 14 different subunits which can be classified by sequence homology into α-type and β-type groups. These subunits are arranged into a barrel-like structure with a length of about 15 nm and a diameter of 11 nm. The central cavity has a diameter of about 5 nm. Within this central cavity is the protease. The active site of the protease is the N-terminal threonine residue of a β-type subunit.

In addition to the core 20S subunit are the accessory proteins that form caps on the ends of the cylinder. This cap subunit forms a 19S structure. The 19S cap is in turn composed of about 20 subunits of which six are ATPases. These accessories confer ATP-dependence to the proteasome and are probably involved in the recognition of the specific substrates, the denaturation of the substrate and the treading of the substrate into the catalytic core. It is this cap structure that confers control over the proteasome. Most of the cap proteins have been identified and cloned.

There is strong homology in all eukaryotes among these cap proteins. Indeed, yeast knock-outs of the ATPases can b e reconstituted with the mammalian homologs. The cap structures and the 20S core together form the 26S proteasome. In all, t h e proteasome contains around 33 different polypeptide subunits ranging in size from 22 to 110 kDa. Proteasomes are present in the nucleus and cytoplasm of the cell and are surprisingly abundant, comprising about 1% of the total cellular protein. It is this abundance that has helped in the isolation and cloning of these many subunit proteins.

Part of the specificity of the proteasome for substrate recognition comes from the ubiquitin system. Most proteins destined for degradation by the proteasome must first be tagged for degradation by the covalent addition of multiple ubiquitin peptides to the ε-NH₂ group of a lysine residue in the protein substrate chain. The conjugation of ubiquitin, a highly conserved 76-amino acid peptide, to the protein is accomplished by a three step reaction. Another source of specificity may be conferred by the subunits of the 19S regulatory unit. In yeast, knock-outs of genes encoding specific subunits of the 19S regulator result in alterations in the degradation of only a subset of proteasome substrates. This feature provides yet another level of control of substrate selection for degradation. Thus, proteasome control is thought to be mediated by the combined selectivity of the ubiquitin system and the 19S cap allowing for both constitutive and signal-dependent degradation of specific proteins that subserve diverse vital roles in the cell.

Proteasomes not only rids the cell of oxidized and otherwise denatured proteins, but are involved in the maintenance of the level of certain proteins that control fundamental processes in the cell. For example, the cyclins, p53 and β-catenin are synthesized at a relatively constant rate. However, their levels in the cell can b e modulated because of the degradation of these proteins by the proteasome. Failure to degrade these and other proteins would lead to their accumulation and profound effects on cellular functions such as cell cycle progression, apoptosis, and developmental gene expression.

The aggregates in Alzheimer's disease and the other neurodegenerative conditions contain ubiquitin. In addition, the proteins that aggregate in the lesions are cleared by the ubiquitin-proteasome system. Proteasomal function has been shown to b e decreased in aging in general and in Alzheimer's disease in particular. Finally, inhibition of proteasomal function by the expression of abnormal ubiquitin leads to neuronal cell apoptotic death. These observations have led to the conclusion that part of the pathogenic mechanism of neurodegeneration might result from the impairment of proteasomal function. Degradation by the proteasome may be inhibited, not only for the proteins that aggregate, but also for the other protein substrates that must be disposed of so that the nerve cell can survive.

These observations have given rise to the most fundamental question about these diseases: do the protein aggregates inhibit the proteasome or do these aggregates form because something else inhibits the proteasome? The answer may differ depending on the neurodegenerative disease, but for Alzheimer's disease, this “chicken or egg” question is hotly debated. For Alzheimer's disease, the amyloid hypothesis suggests that the aggregates (β-amyloid) themselves inhibit the proteasomes. Indeed, for early onset Alzheimer's disease, where mutations in the amyloid precursor protein exist, the primary event is likely to be β-amyloid excess. But for the much more common late onset Alzheimer's, the question still remains: what is the inciting event that starts the process of proteasomal inhibition?

Recently, three groups reported that a region on chromosome 10q confers an inherited susceptibility to late onset Alzheimer's disease or Aβ accumulation (Bertram et al., 2000; Ertekin-Taner et al., 2000; Myers et al., 2000). This work not only verifies the view that such a genetic linkage exists, but also provides a gene locus of interest. Two markers, D10S583 (10q23.33) and D10S1671 (10q25.1) displayed the highest LOD score using a dominant model of inheritance for late onset Alzheimer's disease (Bertram et al., 2000). With respect to location, the O-linked N-acetylglucosamine (O-GlcNAc)-selective N-acetyl-β-D-glucosaminidase (O-GlcNAcase) gene at 10q24.32 is almost exactly halfway between the above mentioned markers. Thus, the location of the O-GlcNAcase gene is appropriate, raising the possibility that protein modification by O-GlcNAc that involves O-GlcNAcase may play a role in late onset Alzheimer's disease.

Glucosamine Metabolism

Glucosamine (GlcN) is synthesized from fructose-6phosphate as a result of the transfer of the amide group from glutamine to the phospho-sugar by the enzyme, glutamine:fructose-6phosphate amidotransferase (GFAT). Glutamine:fructose-6-phosphate amidotransferase is the rate limiting step in GlcN synthesis and there now appears to be two genes that encode this enzyme. The GFAT1 enzyme (the GFAT2 gene is homologous to glutamine:fructose-6phosphate amidotransferase but enzymatic activity has not been published for this protein yet) is tightly regulated in eukaryotic cells.

It has been estimated that only 2-5% of the fructose-6phosphate generated at an early step in glycolysis is diverted into the synthesis of glucosamine. One reason glucosamine synthesis is limited is that the eukaryotic form of glutamine:fructose-6-phosphate amidotransferase is feedback inhibited by a downstream product of glucosamine, UDP-GlcNAc. In eukaryotes, UDP-GlcNAc is ultimately used as the substrate of glycosyltransferases for the synthesis of glycoproteins and glycosaminoglycans. Since the intracellular concentration of UDP-GlcNAc is controlled both by its rate of synthesis and its rate of consumption, the negative feedback on glutamine:fructose-6-phosphate amidotransferase activity by UDP-GlcNAc is then regulated in a manner that corresponds to the metabolic needs of the cell for UDP-GlcNAc. For a rapidly anabolic cell, where glycoprotein synthesis is required for cell growth, the activity of GFAT can be turned on as a result of the consumption of UDPGIcNAc. It has also been shown that the expression of the glutamine:fructose-6-phosphate amidotransferase gene can b e increased in the cell under conditions of growth stimulation. These results suggest that glutamine:fructose-6-phosphate amidotransferase gene transcription may be regulated in a cell growth-dependent fashion such that the amount of enzyme protein is coupled to the level of macromolecular synthesis in the cell. A corollary of this observation is that the system is tuned to avoid excessive glucosamine synthesis.

GFAT can also be regulated by cAMP. Recombinant human glutamine:fructose-6-phosphate amidotransferase was shown to be phosphorylated at two serine residues, ser²⁰⁵ and ser²³¹, by cAMP-dependent protein kinase (A-kinase) in vitro. Stoichiometric phosphorylation resulted in the complete inhibition of GFAT activity. This result implies that glutamine:fructose-6-phosphate amidotransferase activity can be regulated by hormones though cAMP to prioritize F-6-P metabolism to support the energy needs of the intact animal by directing glucose metabolism towards energy production rather than protein glycosylation and macromolecular synthesis.

In summary, UDP-GlcNAc levels reflect the nutritional status of the organism. The UDP reflects the tri-nucleotide pool, the glucose moiety reflects the carbohydrate pool, the amide in GlcN reflects the amino acid pool and the acetate in GlcNAc reflects the pool of acetyl coenzyme A that is derived from metabolites including lipids. Finally, the rate of synthesis of UDP-GlcNAc is feedback and hormone controlled.

O-linked N-acetylglucosamine (O-GlcNAc)

The majority of the glucosamine synthesized in the cell is destined for glycoprotein synthesis. Quantitatively, most glycosylation occurs on those proteins destined for export or the cell surface. This type of glycosylation is initiated as the protein is being translated in the rough endoplasmic reticulum mostly through the N-linkage of complex sugar groups to asparagine residues in the protein. These N-linked complex sugar chains are then modified further in the Golgi apparatus prior to the trafficking of these proteins to the plasma membrane or to exocytotic vesicles.

In the metozoan cells of plants and animals, there is also a form of glycosylation that involves the O-linkage of the monosaccharide, GlcNAc to serine or threonine residues in the protein backbone. The modification is catalyzed by O-GlcNAc transferase (OGT). The OGT cDNA has been cloned and the expression of this enzyme is ubiquitous, although it appears to be most highly expressed in the pancreas, brain and pituitary. The domain structure of O-GlcNAc transferase has been investigated and described. Near the N-terminus, there are 11 tetratricopeptide repeats (TPR), a motif that is involved in protein-protein interactions. In the C-terminal half of the molecule are the domains that bind the UDP and sugar moieties. O-GlcNAc transferase is localized in both the cytoplasm and nucleus and hence, the O-GlcNAc modification can be found on both nuclear and cytoplasmic proteins. This modification also can occur co-translationally, but is highly dynamic. For some proteins, it has been shown that the half-life of the O-GlcNAc is shorter than the half-life of the protein, implying that the O-GlcNAc can be removed and added to proteins post-translationally in a manner similar to phosphorylation. Extracellular signals and the cell cycle have been shown to alter the pattern of proteins modified by O-GlcNAc.

The O-linked N-acetylglucosamine Modification Cycle And Neuro-Endocrine Cell Death

The O-GlcNAc transferase (OGT) gene is most highly expressed in the pancreas, brain and pituitary. In cells with high level of O-GlcNAc transferase expression, blockage of N-acetyl-β-D-glucosaminidase (O-GlcNAcase), which cleaves O-linked N-acetylglucosamine off protein, results in unopposed O-GlcNAc transferase activity that would cause cellular pathology. In situ hybridization showed that pancreatic β-cells have the highest level of O-GicNAc transferase mRNA expression of all known cells. Pancreatic β-cells undergo glucose-dependent apoptosis following blockage of O-GlcNAcase (Liu et al., 2000). Another site of high expression of O-GlcNAc transferase is the somatotropes (growth hormone (GH) secreting cells) in the pituitary. Blockage of O-GlcNAcase resulted in an immediate blunting of GHrelease from and a marked retention of GH secretory granules in the pituitary. These results suggest that O-GlcNAcase blockade in the endocrine tissues where O-GlcNAc transferase is abundant results in a defect in vesicular traffic. Since synaptosomes in the brain contain OGT and O-GlcNAcase, it remains possible that these enzymes play some role in vesicular traffic in the brain. Thus, a defect in O-GlcNAcase may result in a functional defect in neurotransmitter release that could lead to a phenotypic response even before brain cell apoptosis supervenes.

In situ hybridization on brain slices indicated that the hippocampus and Purkinje cells in the cerebellum contain the highest levels of O-GlcNAc transferase mRNA. The O-GlcNAcase mRNA was also abundant in these cells. The hippocampus is a very important region of the brain for the laying down of short term memory and as a site for the development of the pathological changes of Alzheimer's disease. Combining accumulating evidence that the O-GlcNAc pathway plays a role in apoptosis and vesicular traffic with the intriguing genetic evidence of placing the O-GlcNAcase gene at a potential Alzheimer's susceptibly locus on chromosome 10q strongly implicates the O-GlcNAc pathway in neurodegeneration.

The prior art is deficient in methods of modulating the O-GlcNAc pathway in the treatment for neurodegenerative diseases and diabetes. The present invention fulfills this longstanding need and desire in the art.

### SUMMARY OF THE INVENTION

The present invention shows that uncontrolled O-linked protein glycosylation is involved in the pathogenesis of neurodegenerative diseases and diabetes. The O-GlcNAc transferase, an enzyme that mediates O-linked protein glycosylation, is most highly expressed in the pancreas, brain and pituitary. In cells with high level of O-GlcNAc transferase expression, blockage of N-acetyl-β-D-glucosaminidase (O-GleNAcase), which cleaves O-linked N-acetylglucosamine off protein, results in unopposed O-GlcNAc transferase activities that cause cellular pathology.

The present invention indicates that the initial event in late onset Alzheimer's disease is an inherited impairment in the removal of O-linked N-acetylglucosamine (O-GlcNAc) from an ATPase in the 19S cap of the proteasome. The O-GlcNAc modification of the Rpt2/S4 subunit of the proteasome inhibits proteasomal function leading to the accumulation of aggregates that further impair function. The defect in disposal of proteins with a propensity to aggregate results in the pathological observation of inclusion bodies. The defect in the proteasome also results in the accumulation of pro-apoptotic proteins that are normally held at very low concentration in the cell by the proteasome. A failure to degrade these toxic proteins signals the apoptotic death of neurons. It is the loss of neurons in critical areas of the brain that leads to the clinical manifestations of the neurodegenerative disorder.

These studies on the control of proteasome function by O-GlcNAc modification of the Rpt2/S4 ATPase subunit of the proteasome has linked the metabolic state of a cell to the activity of proteasome itself. This research is the first example of an endogenous regulator of proteasomal activity. The activity of the O-GlcNAc transferase, the enzyme that modifies proteins, depends on the concentration of UDP-GlcNAc. Since the level of UDP-GlcNAc, the substrate for O-GlcNAc transferase, reflects availability of trinucleotides, glucose, acetylcoenzyme A and amino acids, the activity of the proteasome and neuronal function may be directly modulated by the availability of nutrients. This raises a tantalizing connection between nutritional deprivation, augmented proteasomal activity (less O-GlcNAcylation) and prolongation of life. Because the O-linked N-acetylglucosamine pathway is well defined, drugs may be developed that can partially block this pathway as a novel approach to the prevention of neurodegeneration and even aging itself.

Results presented herein also implicate O-glycosylation in the pathogenesis of diabetes. Streptozotocin (STZ), an analog of N-acetylglucosamine (GlcNAc), is a specific toxin for the pancreatic β-cell. Streptozotocin has been demonstrated to act by inhibiting the enzyme O-GlcNAcase, which cleaves O-linked N-acetylglucosamine off protein. Both glucose and the diabetogenic compound streptozotocin stimulate O-glycosylation on a protein of 135 kDa in pancreatic islets (Konrad et al., 2000, 2001a, 2002). Recently, this 135 kDa protein was identified as O-GlcNAc transferase itself (Konrad et al., 2001b). The present invention shows that it is possible to pharmacologically block this process of O-glycosylation from occurring. In one embodiment of the present invention, the molecular structure of the compound (Z)-1[N-(3-Ammoniopropyl)-N-(n-propyl)amino]diazen-ium-1,2-diolate (also known as PAPA NONOate or NOC-15) is provided and compared to the structures of similar compounds that do not inhibit O-glycosylation, indicating what structural aspects are necessary for inhibition of protein O-glycosylation.

In another embodiment of the present invention, the concept of using NOC-15, NOC-15-related molecules, other structural analogs of N-acetylglucosamine, or other related molecules to inhibit O-linked protein glycosylation in tissues other than pancreatic beta-cells is proposed, with the idea that O-glycosylation may be an important pathway in other disease processes and that inhibition of this pathway may be of great clinical utility.

### BRIEF DESCRIPTION OF THE DRAWINGS

FIG. 1 shows SpI degradation was inhibited b y vaccinia virus-expressed O-GlcNAc transferase (OGT). Activated nuclear extract was pre-treated with or without GST, GST-OGTN286, GST-OGTN485 or GST-OGT beads at room temperature for 30 min. The beads were spun down and discarded. Sp1 was then added to the supernatant and incubated at room temperature for 45 min. SDSPAGE and anti-GST blots were performed. UDP-GalNAc partially inhibits O-GlcNAc transferase.

FIG. 2 shows proteasome chymotryptic activity a s measured in a fluorescence assay. Nuclear extract (NE) was pr etreated with or without proteasome-specific inhibitors: β-lactone, PSI, LlnL or epoxomicin and GST or GST-OGT beads at room temperature for 30 min. The beads were spun down and discarded. Fluorogenic peptide substrate suc-LLVY-AMC was then added to the supernatnat and incubated at 37° C. for 2 hours. Fluorescence was visualized with 365 nM UV.

FIG. 3 shows the chymotryptic activity of the 26S proteasome was inhibited by O-GlcNAc transferase as determined in the suc-LLVY-AMC assay.

FIG. 4 shows the chymotryptic activity of the 20S proteasome was not inhibited by O-GlcNAc transferase as determined in the suc-LLVY-AMC assay.

FIG. 5 shows the ATPase activity of purified 26S proteasomes as measured by the liberation of inorganic phosphate from ATP. The gray and black bars indicate ATPase activity before and after treatment with UDP-GlcNAc (group 1); GST, UDP-GlcNAc (group 2); or GST-OGT, UDP-GlcNAc (group 3).

FIG. 6 shows O-linked protein glycosylation of purified proteasomes treated with OGT, UDP-GlcNAc. The Rpt2/S4 ATPase subunit of the 19S cap of the proteasome was precipitated with Rpt2/S4 antibodies, and the level of O-GlcNAc modification was assessed by western blot with RL2 antibody specific for O-GleNAc. Left lane: no OGT; right lane: OGT added.

FIG. 7 shows proteasomes were treated with GST-OGT as before, and all the proteasome proteins were run on a gel. Only one protein with the molecular weight of Rpt2/S4 changes its state of glycosylation.

FIG. 8 shows the structures of streptozotocin (STZ), alloxan (ALX) and UDP-N-acetylglucosamine (UDP-GlcNAc).

FIG. 9 shows the molecular structures of NOC-15, and N-Acetylglucosamine (GlcNAc). It is apparent from the structures that NOC-15 is an analog of N-Acetylglucosamine.

FIG. 10 shows what occurs if islets were stimulated with 3 mM glucose (lane A), 5 mM streptozotocin (lane B), 5 mM NOC-15 (lane C) or 5 mM DPTA (lane D). After immunoprecipitation and Western blotting of O-glycosylated protein with RL2 antibody, p135 was shown to undergo increased O-glycosylation in response to STZ.

FIG. 11 shows NOC-15 completely abolishes p135 O-glycosylation. Islets cells were stimulated with 3 mM glucose (lane A), 5 mM streptozotocin (lane B), or 5 mM streptozotocin and 5 mM NOC-15 (lane C). After immunoprecipitation and Western blotting of O-glycosylated protein with RL2 antibody, p135 is shown to undergo increased O-glycosylation in response to streptozotocin. The addition of NOC-15, even in the presence of streptozotocin is able to almost completely abolish p135 O-glycosylation.

FIG. 12 shows the molecular structures of NOC-15, and the similar molecules DPTA, and Spermine NONOate. In contrast to NOC-15, DPTA and Spermine NONOate do not inhibit p135 O-glycosylation, indicating that the terminal methyl group present in NOC-15 may be critical for its ability to inhibit O-glycosylation.

FIG. 13 shows pancreatic islets cells examined by in situ TUNEL assay (upper panels) and western blot with anti-p53 (lower panels).

FIG. 14 shows β-gal staining of islets at indicated times after treatment of the animals with streptozotocin. Upper panel, TGFα-βgal reporter mice; lower panel, same reporter mice with homozygous p53 knock-out.

FIG. 15 shows p53 accumulation in brain after intracerebroventricular injection of streptozotocin. The reporter mice were injected with vehicle (upper panel) or streptozotocin (lower two panels) and the hippocampal neurons were stained for p53. The brain from a homozygous p53 knock-out mouse showed no staining (lower panel).

FIG. 16 shows β-gal staining in brain after intracerebroventricular injection of streptozotocin. The animals were treated as described in FIG. 15.

FIG. 17 shows RL2 staining of hippocampal neuron before and 6 hours after intracerebroventricular injection of streptozotocin.

FIG. 18 shows LLVY cleavage can be inhibited when OGT is added to NRK nuclear extract (upper panel). Beta-lactone inhibits LLVY cleavage indicating that the activity is proteasomal. There is less activity in the brain of STZ-treated animal.

FIG. 19 shows a schematic showing the tetracycline transgene system. The brain specific promoter will be the PDGF-β chain promoter as used in the PDAPP mouse.

FIG. 20 shows a pool of 3T3 cells were cotransfected with the transgene (TetRE-O-GlcNAcase(AS)), activator (CMV-rtTA) and transfection indicator (CMV-β-Gal). Cells were plated on a chamber slide and following recovery, treated with or without the inducer, doxycycline. The cells on the slide were stained red for β-gal and green for O-GleNAc content with RL2 Ab. The induced transgene increased cellular O-GlcNAc content.

### DETAILED DESCRIPTION OF THE INVENTION

The present invention provides evidence that support a role of O-linked protein glycosylation in neuro-endocrine cell death that leads to neurodegenerative diseases and diabetes. Consequently, inhibition of O-linked protein glycosylation by inhibitors such as structural analogs of N-acetylglucosamine or other related molecules may be employed in new approaches to prevent and treat these diseases.

O-Linked Protein Glycosylation and Neurodengenerative Diseases

The neurodegenerative disorders are widely believed to develop as a result of a problem in the clearance of toxic proteins from the nerve cells by proteasomes. These toxic proteins are thought to be normal constituents of the apoptotic signal whose clearance from the cell by the proteasome is necessary for cell survival. Failure t o clear these proteins leads to the apoptotic death of the neuron. In some cases, not only are the characteristic aggregates of these diseases cleared by the proteasome, but these aggregates impede proteasomal function thereby leading to the self-perpetuation and amplification of the disease.

For late onset Alzheimer's disease, where aggregate proteins are not overproduced, it remains unclear why they begin to accumulate in the first place. The present invention shows that the 26S proteasome can be inhibited when one of the ATPases in the 19S cap is modified by the O-linkage of N-acetylglucosamine (O-GlcNAc). The enzyme that places this modification on this subunit is O-GlcNAc transferase while the enzyme that removes it is the O-GlcNAcase. The O-GlcNAcase can be non-competitively inhibited by the GlcNAc-like antibiotic, streptozotocin.

The position and function of the O-GlcNAcase gene make it a promising candidate gene for late onset Alzheimer's disease. Recent studies have identified a promising gene for late onset Alzheimer's disease on chromosome 10 between 10q23.33 and 10q25.1. The STZ-inhibitable O-GlcNAcase gene is at 10q24.32. Any impairment of O-GlcNAcase function would result in the modification of more proteasomes by O-GlcNAc. This modification could start the spiraling cascade of aggregate accumulation and protosomal inhibition proposed as central to the pathogenesis of Alzheimer's disease.

O-Linked Protein Glycosylation and Diabetes

Streptozotocin, an analog of N-acetylglucosamine (GlcNAc), is a specific toxin for the pancreatic β-cells. Streptozotocin has been demonstrated to act by inhibiting the enzyme O-GlcNAcase, which cleaves O-linked N-acetylglucosamine off protein. When administered to rats or other animals, streptozotocin causes diabetes. Treatment of rats with streptozotocin also results in vivo in an early β-cell-specific increase in the level of intracellular protein modification by O-linked N-acetylglucosamine (O-GIcNAc). Treatment of isolated islets with streptozotocin in vitro results in increased O-glycosylation of a protein called p135. High levels of glucose also have the same effect on p135. The pancreatic beta-cell is likely exquisitely sensitive to streptozotocin because it contains 100-1000 fold more of the enzyme O-linked N-acetylglucosamine transferase than any other cell type.

Since streptozotocin causes diabetes and glucose shares with streptozotocin effect of increased p135 O-glycosylation, it is very likely that the way in which diabetes occurs is that high levels of glucose present early in the course of pre-diabetes are toxic to pancreatic beta-cells in the same manner as streptozotocin. This leads to beta-cell failure, which leads to higher levels of glucose, which leads to more beta-cell failure, etc. Eventually, enough beta-cells are damaged/destroyed that full blown diabetes develops. Therefore, in order to prevent and/or treat diabetes, it is necessary to inhibit pancreatic beta-cell O-linked protein glycosylation.

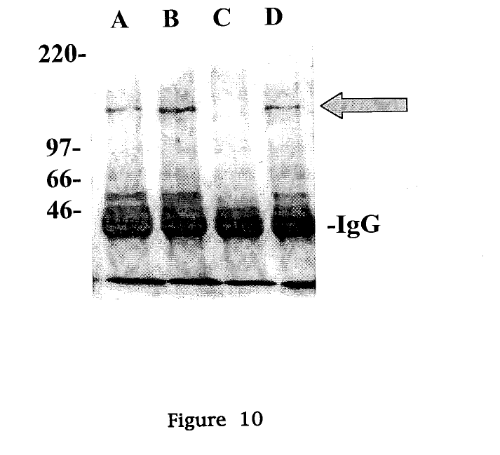

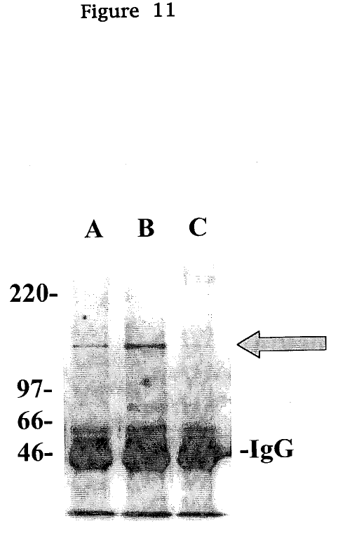

This can be accomplished by using the drug NOC-15, which is a structural analog of N-acetylglucosamine, the natural substrate of O-linked N-acetylglucosamine transferase. As can clearly be seen from FIGS. 10 and 11, NOC-15 was able to inhibit pancreatic beta-cell p135 O-glycosylation, even when the islets were treated with 5 mM streptozotocin. The ability of NOC-15 to counteract even the effect of 5 mM streptozotocin suggests that almost complete inhibition of O-glycosylation is possible. This data thus suggests a possible pharmacologic approach for preventing or treating diabetes.

NOC-15 is a nitric oxide donor and thus probably acts by binding to O-linked N-acetylglucosamine transferase and giving off at least one (and possibly 2) molecule(s) of nitric oxide in the active site of the enzyme, thus inactivating O-linked N-acetylglucosamine transferase and abolishing the process of O-linked protein glycosylation. Obviously, because it acts as a nitric oxide donor, NOC15 is probably not suitable for human use. However, by modifying its basic structure, a person having ordinary skill in this art would be able to synthesize an inhibitor of O-linked N-acetylglucosamine transferase that does not have the undesirable side effect of acting as a NO donor.

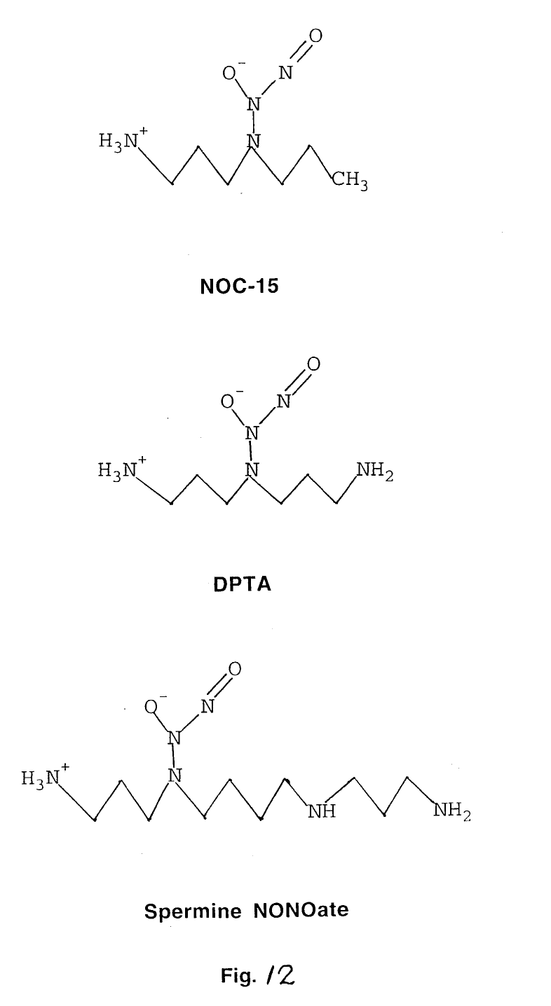

FIG. 12 also shows that the methyl group present at the end of the side chain is essential for NOC-15's activity. The related molecules DPTA NONOate and Spermine NONOate, which differ only slightly in structure with respect to this methyl group, do not have the ability to inhibit O-linked protein glycosylation. Thus, by using the information contained herein, it would be possible to synthesize a non-toxic inhibitor of O-linked protein glycosylation and such a n inhibitor should prove extremely useful in preventing and/or treating diabetes mellitus.

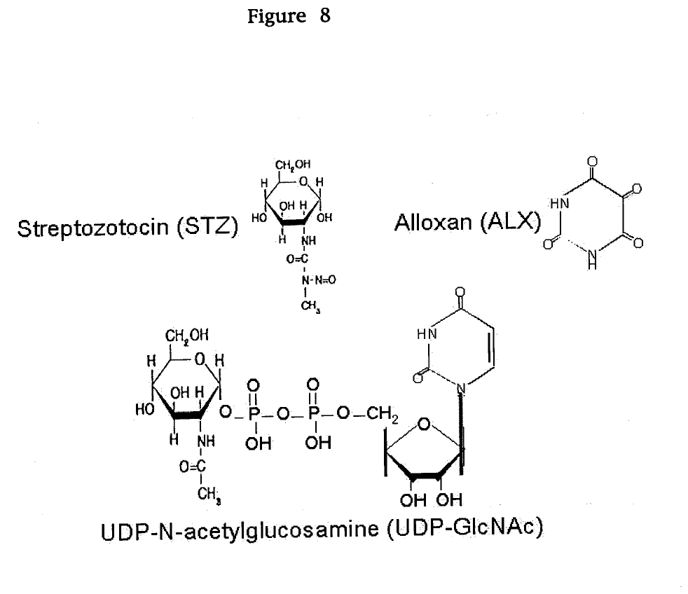

Another chemical means of inhibiting O-GlcNAc transferase has been discovered. Another long known diabetogenic drug, alloxan (ALX) (FIG. 8 above), is a uracil analog. Injections of this drug into laboratory rats also causes a relatively specific death of the pancreatic β-cells. Studies of the dying β-cells indicates that the cells do not die of apoptosis as they do after treatment with STZ. Isolated β-cells treated with alloxan fail to display an increase in the O-GlcNAc modification of p135 in response to glucose or glucosamine. Purified recombinant O-GlcNAc transferase is inhibited half-maximally by 0.1 mM alloxan, compatible with the dose of this drug that causes diabetes. These studies indicate that an analog of UDP-GlcNAc that mimics the uracil moiety like alloxan, might be an index compound for the discovery of other UDP-GlcNAc like molecules that inhibit the O-GlcNAc transferase.

As used herein, the term ‘O-linked protein glycosylation’ refers to glycosylation that involves the O-linkage of N-acetylglucosamine (GlcNAc) to serine or threonine residues in the protein backbone. The enzyme performing this protein modification is O-linked N-acetylglucosamine transferase.

Thus, the present invention is drawn to a method of inhibiting neuro-endocrine cell death by inhibiting the enzymatic activity of O-linked N-acetylglucosamine transferase. Inhibition of O-linked protein glycosylation resulted from inhibition of said enzyme would lead to prevention of neuro-endocrine cell death. Preferably, the enzyme inhibitor is a structural analog of N-acetylglucosamine including but not limited to (Z)-1-[N-(3-Ammoniopropyl)-N-(n-propyl)amino]diazen-ium-1,2-diolate (NOC-15) and derivatives thereof and uracil-like drugs, exemplified but not limited to compounds like alloxan. In general, the neuro-endocrine cells include pancreatic β-cells, cells of the central nervous system including the brain and spinal cord and other hormone-secreting cells including the pituitary gland.

In another embodiment of the present invention, the concept of using NOC-15, NOC-15-related molecules, other structural analogs of N-acetylglucosamine, or other related molecules to inhibit O-linked N-acetylglucosamine transferase is proposed as a method for the prevention and/or treatment of diabetes and late onset of Alzheimer's disease.

The following examples are given for the purpose of illustrating various embodiments of the invention and are not meant to limit the present invention in any fashion. One skilled in the art will appreciate readily that the present invention is well adapted to carry out the objects and obtain the ends and advantages mentioned, as well as those objects, ends and advantages inherent herein. Changes therein and other uses which are encompassed within the spirit of the invention as defined by the scope of the claims will occur to those skilled in the art.

### EXAMPLE 1

Direct Effect of O-linked N-acetylglucosamine Modification on Transcription

Transcriptional activation essentially results from DNA-directed protein-protein interactions. Such interactions had been worked out well for transcription factor Sp1. Domain B of Sp1 had been shown to confer the ability of Sp1 to homo-multimerize and to interact with the TATA-binding protein associated factor, termed TAF110. Using a portion of Sp1 that confers these interactions, direct pull-down experiments were performed with glycosylated and unglycosylated peptide. The results showed that the interactions involving this Sp1 peptide were blocked by O-GlcNAc. Subsequently, it was confirmed that the in vivo interaction between holo-Sp1 and the SpE peptide is blocked by O-GlcNAc.

As predicted from these interaction studies, glycosylation of the Sp1 transcriptional activation domain blocked transcriptional activation in nuclear extracts using in vitro transcription assays. Indeed, overexpression of O-GlcNAc transferase in cells suppressed transcriptional activation stimulated by holo-Sp1 and a Gal4-Sp1 fusion protein. This difference in transcriptional activation can also be observed in pancreatic β-cells, a cell type that naturally overexpresses O-GlcNAc transferase (see below).

Because the O-GlcNAc modification blocks Sp1-mediated gene activation, and Sp1 plays so broad a role in transcription, it was investigated whether O-GlcNAc transferase may be part of a corepressor complex, hence playing a very general role in the repression of gene expression. It was shown that functionally O-GlcNAc transferase and mSin3A cooperatively repressed transcription in parallel with histone deacetylation. It was proposed that mSin3A targeted O-GlcNAc transferase to specific promoters to inactivate transcription factors and RNA polymerase II by O-GlcNAc modification, which acted in concert with histone deacetylation to promote gene silencing in an efficient, concerted and specific manner. The involvement of O-GlcNAc transferase in the repression complex appears to be very general. There are evidences this pathway also represses genes in the Wnt signaling pathway via the Groucho-related transcriptional repressor, TLE1 and TLE2.

This profound difference in behavior of a transcriptional activation domain depending on whether the domain is modified b y O-GlcNAc or not is the first direct demonstration that O-GlcNAc can directly change the function of a transcription factor and that this change of function is not related to phosphorylation. Based on findings so far that the O-GlcNAc transferase TPR motif recognizes hydrophobic domains in proteins, it is likely that modification by O-GlcNAc of these hydrophobic regions in O-GlcNAc transferase substrates results in a change in the degree of hydrophobicity in the protein interaction domain, thus disrupting protein-protein interactions.

These studies suggest that if O-GlcNAc transferase is involved in co-repression, then the converse enzyme, O-GlcNAcselective N-acetyl-β-D-glucosaminidase (O-GlcNAcase) may be involved in the activation of gene expression. O-GlcNAcase has been detected in nuclear extract; however, there are no studies to show how this activity is controlled. The cloning of the O-GlcNAcase has been reported and the inventors have independently cloned the rat and mouse cDNAs and splice variants (accession number AY039679). The mouse form is active when expressed either in E. coli or vaccinia virus. The mouse form will be used for a transgenic model a s described below.

### EXAMPLE 2

O-Linked N-Acetylglucosamine Modification and Protein Degradation

It has been shown that cells exposed to conditions of low glucose and cAMP accumulation displayed a rapid depletion of Sp1 DNA-binding activity and protein. This loss of Sp1 could be blocked by the highly specific inhibitors of proteasomes, MG132 and lactacystin and also by LLnL, suggesting that Sp1 was subject to proteasome degradation. In experiments where proteasome degradation of Sp1 was blocked by lactacystin, it was shown that treatment with cAMP and low glucose resulted in near total loss of 0GlcNAc residues from the Sp1 protein and other proteins in the cell. This study therefore correlated the stability of Sp1 against proteasome degradation with the level of Sp1 modification by O-GleNAc.

The depletion of O-GlcNAc on Sp1 and other proteins appeared to have resulted both from the glucose starvation and from the cAMP. The glucose starvation probably lowered F-6-P levels while the cAMP blocked the activity of GFAT. When the cells were provided glucosamine following glucose starvation and forskolin, Sp1 glycosylation was increased and degradation decreased.

These findings have two implications. The first is that the amount of O-GlcNAc on Sp1 can be modulated by changes in extracellular glucose concentrations and by signals that change cAMP levels. The second implication is that the proteasomal degradation of Sp1 might be part of a nutritional sensing system. That is, when cells are under nutritional stress (glucose starvation), Sp1, which controls the transcription of most housekeeping genes, is degraded. The consequence of Sp1 degradation by the proteasome might be to block macromolecular synthesis to conserve nutrients under nutritional stress situations. The stabilization of Sp1 or other transcription factors resulting from O-GlcNAc blockade of proteasomes may increase transcription of many genes. If proteasome blockade is more general, then broader implications to the cell may result. As the following results will show, there are indeed broader effects of O-GlcNAc on the proteasome. Data presented below suggest that the inhibition of the proteasome by O-GlcNAc may result in apoptosis and aggregate formation, and the linkage of a nutrient metabolic pathway to the control of the proteasome could provide a site for intervention by drug therapy to prevent neuro-endocrine cell death that leads to neurodegeneration and diabetes.

### EXAMPLE 3

Direct Regulation of Proteasome by O-Linked N-Acetylglucosamine

A reconstituted in vitro system has now been developed using recombinant Sp1 to define the domains in Sp1 that are involved in the proteasome degradation process. Recombinant Sp1 and deletion mutants of Sp1 expressed using a vaccinia virus system were tagged on the N-terminus with glutathione-S-transferase (GST) to facilitate its purification. The degradation of Sp1 by a proteasome-dependent mechanism could be reproduced by exposing the recombinant Sp1 to nuclear extracts. However, degradation is only observed if the nuclear extract is derived from cells that had been glucose-starved and forskolin-stimulated (cAMP). This extract is termed activated extract. Conversely, cells treated with glucosamine prior to the harvest of the nuclear extract yield an extract that is incapable of processing the Sp1. The degradation is dependent on ATP and can be inhibited by proteasome inhibitors added to the intact cells prior to the nuclear extract preparation or if the inhibitor is added to the nuclear extracts directly.

The N-terminal domain of Sp1 has been identified as the target domain for proteasome-dependent degradation. The proteasome cleaves this domain of Sp1 at a site downstream of a glycine-rich region (GRR). A similar region has been observed to play a role in the proteasomal processing of the 105 kDa precursor of NF-κB. The major portion of the protein downstream of this Leu⁵⁶ cleavage site is degraded by the proteasome. The N-terminal target sequence of Sp1 appears to be necessary and sufficient for proteasomal targeting. When the N-terminal segment of Sp1 is fused to a heterologous protein, the same cleavage is observed. When this segment is deleted from Sp1, proteasomal degradation of Sp1 does not occur efficiently. Finally, when a large excess of the Sp1 N-terminal peptide, termed SpV, is added to the reconstituted system, Sp1 degradation is blocked. This latter observation suggests that this recognition site of Sp1 can saturate a corresponding recognition protein that is involved with the proteasome.

These studies also suggest that the degradation of Sp1 by the proteasome does not depend on the O-GlcNAc status of Sp1 itself, but on the O-GlcNAc status of a protein in the proteasome or nuclear extract. That is, proteasomal degradation of Sp1 or the Sp1 peptide fused to a heterologous protein does not occur in an extract derived from cells treated with glucosamine. Furthermore, the Sp1 targeting sequence is not glycosylated nor is it ubiquitinated. To confirm that it is not the O-GlcNAc state of Sp1 itself that regulates degradation, the inventors have expressed Sp1 in various states of glycosylation in the vaccinia system. It was found that an activated nuclear extract degrades Sp1 regardless of its level of O-GlcNAcylation.

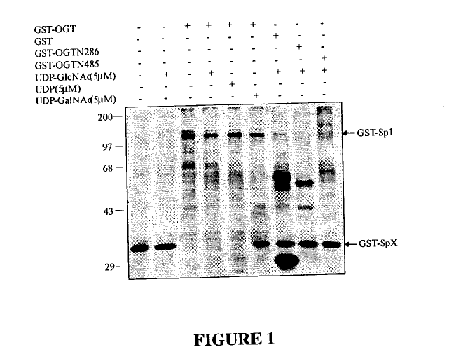

To show definitively that the inhibition of the proteasome processing of Sp1 results from O-GlcNAc modification, the inventors have expressed O-GlcNAc transferase and its catalytically inactive mutants and supplemented activated nuclear extract with these purified enzymes. O-GlcNAc transferase bound to glutathione beads (GST-OGT) was added for 15 minutes to activated nuclear extract. The bead-bound GST-OGT was then removed by centrifugation. The activity of the proteasome was assayed with Sp1 as the substrate. These studies indicated that a brief preincubation of the active nuclear extract with O-GlcNAc transferase resulted in complete inactivation of the proteasome activity to cleave Sp1 and generate SpX (FIG. 1). While no dependence on UDP-GlcNAc was observed in this experiment, likely because the nuclear extract or purified O-GlcNAc transferase contains this substrate, UDP-GalNAc, an O-GlcNAc transferase inhibitor partially blocked the inhibitory effect of catalytically active O-GlcNAc transferase. A variety of O-GlcNAc transferase mutants that lack either the tetratricopeptide repeats (TPRs) or catalytic domains have been developed (Yang et al., 2002). Treatment of extract with these mutants did not inhibit proteasomal activity (Yang et al., 2002). These mutants confirm that the enzymatic activity of O-GlcNAc transferase is necessary for proteasome inactivation and that the O-GlcNAc transferase TPR region does not act independently.

Rat (accession AY039679) and mouse O-GlcNAcase and a variety of splice variants were cloned and the longest mouse O-GlcNAcase was expressed as a GST-fusion protein both in the vaccinia system and in E. coli. The protein has enzyme activity measured using the synthetic substrate, p-nitrophenyl-N-acetyl-β-D-glucosaminide as described previously (Gao et al., 2001; Konrad et al., 2001a). As predicted, treatment of inactive nuclear extract from glucosaminetreated cells with GST-O-GlcNAcase activates Sp1 degradation by the proteasome. The ability to activate the proteasomes in a nuclear extract that had been previously inactivated in vivo by glucosamine further substantiates the hypothesis that there is an O-GlcNAc-protein that reversibly modulates proteasomal degradation of the Sp1 substrate in vivo.

The chymotryptic activity of the proteasome itself can be measured using short synthetic peptides. The peptide, N-succinyl-Leu-Leu-Val-Tyr-7-amido-4-methylcoumarin (suc-LLVY-AMC) (Sigma), is coupled to a fluorescent indicator, and the cleavage can be monitored and quantitated by a fluorometer. These peptides cannot be ubiquitinated (no lysine), hence the measured activity depends only on that of the proteasome and not the more complex ubiquitinization system. The reaction is carried out in a 96 well plate.

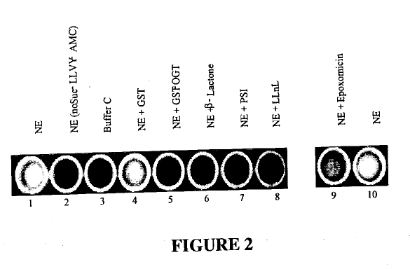

If the chymotryptic cleavage has occurred, the fluorescence can b e excited with UV light at 365 nm and detected by a CCDdigital camera coupled to a computer. An example of this assay system is shown in FIG. 2. Because the LLVY peptide can be cleaved by any enzyme having chymotryptic activity, cleavage is not specifically proteasome-dependent. However, if cleavage is blocked by a wide spectrum proteasome inhibitors, then it is reasonable to deduce that the detected fluorescence is proteasome-dependent.

Proteasome peptidase activity in activated NRK nuclear extracts was examined with this assay. The activity could be inhibited by preincubating the extract with known proteasome inhibitors indicating the the fluorescent signal derives from authentic proteasome activity (FIG. 2). If the cells were treated with lactacystin or glucosamine, the extract from these cells also does not display this activity (data not shown). When the extract was exposed briefly to GST-OGT plus UDP-GlcNAc and then the O-GlcNAc transferase was removed, the chymotryptic activity was eliminated. GST had no effect on the proteasome activity. This experiment demonstrated that not only was Sp1 processing blocked by O-GlcNAc, but the chymotryptic activity of the proteasome itself was inhibited. Inactive NRK extract was also treated with GST-O-GlcNAcase and proteasomal function was assayed before and after this treatment using the suc-LLVY-AMC assay. The chymotryptic activity of the proteasome was stimulated by this treatment.

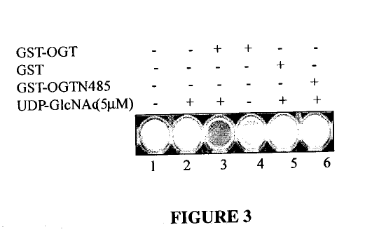

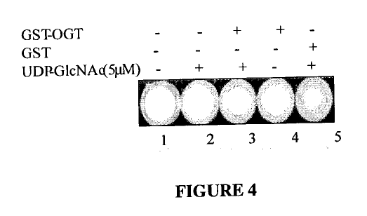

Purified 26S and 20S proteasomes were assayed for activity using the suc-LLVY-AMC assay. With intact 26S proteasomes (FIG. 3), the suc-LLVY-AMC chymotryptic activity was inhibited by bacterially expressed GST-OGT but not GST-OGTN485 (TPR domain with absent C-terminal catalytic domain). Inhibition was dependent on UDP-GlcNAc and catalytically active O-GlcNAc transferase. However, for the 20S proteasome, no inhibition of activity w as observed (FIG. 4).

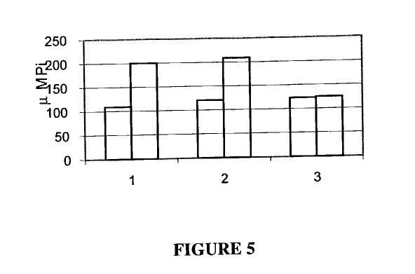

The ATPase activity of the purified 26S proteasomes was measured by detecting the liberation of inorganic phosphate from ATP. Treatment of the proteasomes with UDP-GlcNAc or GST plus UDP-GlcNAc had no effect. However, when enzymatically active GST-OGT was added to the proteasomes with UDP-GlcNAc, the ATPase activity was blocked (FIG. 5).

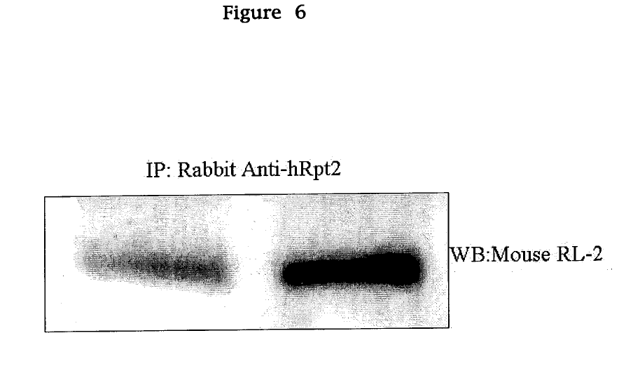

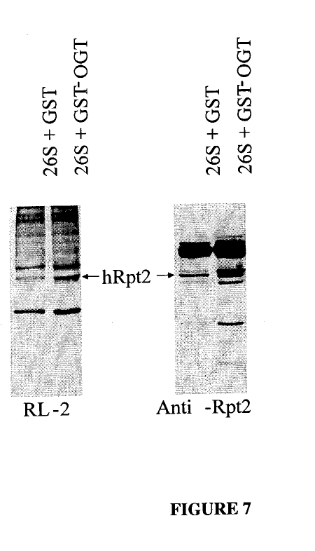

After treatment of the proteasomes with GST-OGT, the proteasomes proteins were disrupted and the Rpt2/S4 ATPase subunit of the 19S cap of the proteasome was precipitated with Rpt2/S4 antibodies. The protein was western-blotted and probed with the RL2 antibody (an antibody specific for O-GlcNAc) to determine if OGT treatment resulted in glycosylation of this subunit. FIG. 6 shows that this subunit is O-GlcNAcylated in the basal state, but OGT increased the signal. This change in the O-GlcNAc level corresponds to the loss of function of the proteasome. If proteasomes were treated with GST-OGT as before, but all the proteasome proteins were run on a gel, then only one protein changes its state of glycosylation. This protein has the same molecular weight as Rpt2/S4 (FIG. 7).

In studies on yeast proteasomes, mutations in the ATP binding sites of the six ATPases results in different phenotypes. Only a mutation of Rpt2 results in a phenotype in which the chymotryptic activity against LLVY is blocked. Thus, the human proteasome shows a strong homology to its yeast counterpart in that O-GlcNAcylation of Rpt2/S4 results in the similar behavior in humans as mutation of Rpt2 in yeast.

The inventors have cloned and expressed human Rpt2/S4 and will determine if it is a good substrate for O-GlcNAc transferase in vitro. If so, the glycosylation site(s) can be mapped allowing the mutation of these sites and further studies to determine if this modification is necessary and sufficient to block proteasomal function.

The finding that O-GlcNAc transferase has the ability to turn off 26S proteasome function centrally through the modification of one of the ATPases is the first observation that proteasome function can be regulated by an endogenous substance at the level of the proteasome. All prior inhibitors of proteasomes are fungal-derived antibiotics or synthetic peptides. The implication is that we appear to have found a means of coupling nutrient metabolism to proteasomal function. Thus, the proteasomes, with its need for ATP-derived energy and its regulation by a nutrient metabolite has a new and unexpected role in energy sensing and homeostasis. This observation is analogous to the proteasomal role in the starvation response in E. coli.

### EXAMPLE 4

Inhibition of Streptozotocin (STZ)-Induced Pancreatic Islet p135 O-Glycosylation

Two diabetogenic drugs, alloxan and streptozotocin (STZ), are inhibitors of the O-GlcNAc cycle of addition and removal of O-GlcNAc streptozotocin is an inhibitor of the O-GlcNAcase, while alloxan is an inhibitor of O-GlcNAc transferase. STZ is chemically related to GlcNAc while alloxan is chemically related to uracil (FIG. 8). These structural features help explain the relative specificity of these inhibitors for their respective enzymes. Kinetic studies using recombinant O-GlcNAcase indicate that streptozotocin is a noncompetitive inhibitor of the enzyme whereas another chemical inhibitor of the O-GlcNAcase, PUGNAC, displays competitive kinetics.

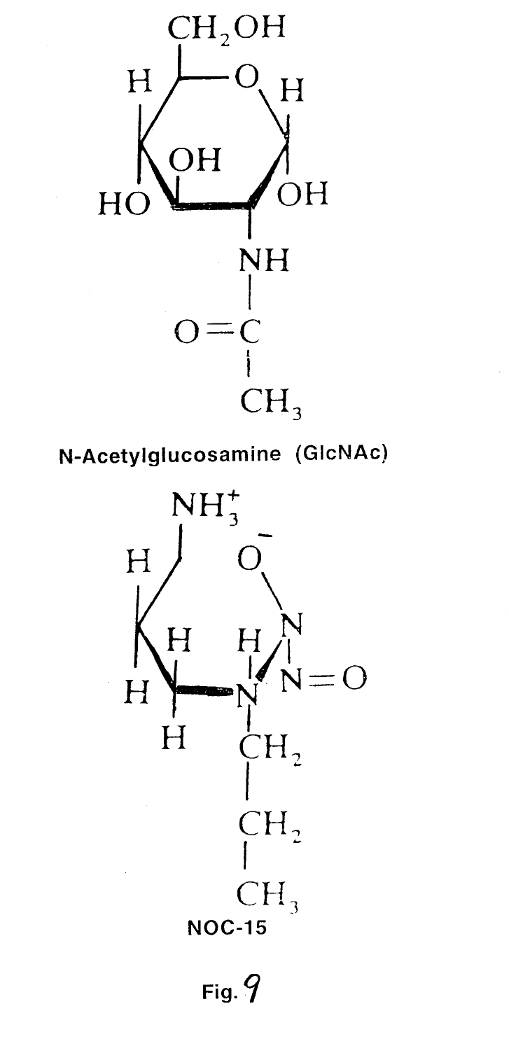

Rat pancreatic islets were isolated, counted into tubes and pre-incubated with 3 mM glucose. Following pre-incubation, islets were stimulated with 3 mM glucose, 5 mM STZ, 5 mM NOC-15, or 5 mM DPTA. NOC-15 ((Z)-1-[N-(3-Ammoniopropyl)-N-(n-propyl)amino]diazen-ium-1,2-diolate, also known as PAPA NONOate) is a structural analog of N-acetylglucosamine (FIG. 9). At the end of the experiment, O-glycosylated proteins were immunoprecipitated with RL2 antibody, which binds to O-linked N-acetylglucosamine. Immunoprecipitated proteins were run out on a 6.5% SDS-PAGE gel and the proteins were then transferred to a nitrocellulose blot. The blot was then probed with RL2 antibody, which was detected by the ECL method. The blot was exposed to X-ray film and the film was developed and photographed. The results shown demonstrate that islets contain a major O-glycosylated protein called p135 and that its O-glycosylation is stimulated by STZ (FIG. 10). The results also indicate that NOC-15 is capable of almost completely inhibiting STZ-induced p135 O-glycosylation (FIG. 11). These data suggest that the pancreatic beta-cell O-glycosylation pathway (which is involved in the development of STZ-induced diabetes) is amenable to pharmacologic therapy. The structure and action of NOC-15 can thus be used as a basis to synthesize drugs that can be used to prevent and/or treat diabetes.

### EXAMPLE 5

TGFα-βgal Reporter Transgenic Mouse Model

This example describes the establishment of the TGFα-βgal transgenic mouse as a reporter of p53 function in the brain. The system will be used to correlate the reporter function to neuronal apoptosis, brain proteasome function and behavioral changes resulting from perturbations of O-GlcNAc metabolism in the brain.

Characterization of The TGFα-LacZ Reporter As An Indicator of p53 Activation

It has been determined that the TGFα promoter contains two p53 elements, one in the distal promoter (˜600 bp upstream of transcriptional start) and one more proximal (˜50 upstream). While p53 was conventionally thought of as a tumor suppressor while TGFα a tumor promoter, p53 is now thought of as an important molecule for cell repair. When the DNA in a cell is damaged by chemicals or radiation, p53 ceases to be degraded by the proteasome. Because its synthesis rate remains constant, while its rate of degradation is markedly reduced, p53 protein accumulates. Other signals in the cell determine whether the cell should die by apoptosis or whether to allow the cell to enter cycle arrest so that it can be repaired. It is postulated that an injured cell in a barrier epithelium such as skin or colon must be replaced by a daughter cell to maintain the integrity of the barrier. Thus, when p53 accumulates, and the choice is made to kill the cell, p53 induces the expression of apoptosis genes. At the same time, p53 would induce the expression of TGFα so that the dying cell could leave behind a growth signal that would ensure its replacement in the barrier.

The TGFα promoter was placed in front of the β-galactosidase reporter gene and developed two lines of transgenic mice. The line with the best reporter activity was used in all subsequent experiments. Tissue expression of the reporter was detected as follows: tissues were quickly excised, cut into pieces, and fixed in 4% paraformaldehyde (made in PBS, pH 7.5) for 30 min at 4° C. Tissue sections were rinsed three times 10 min each in ice-cold PBS, immersed in freshly prepared staining solution [10 ml solution of PBS (pH 7.4) containing 2 mM MgCl₂, 0.01% sodium deoxycholate, 0.02% NP40, 0.1% X-gal dissolved in dimethylformamide, 82.5 mg potassium ferricyanide, and 94.5 mg potassium ferrocyanide], and incubated overnight at 37° C. The reaction was stopped by rinsing the organs in PBS. Sampleswere stored in 70% ethanol at 4° C. In validation studies, the areas where native TGFα are expressed showed good reporter activity. For example, β-gal activity was detected in the pituitary and in some neurons, sites where native TGFα had been detected before. Similarly, sites where native TGFα is not detected did not show β-gal reporter activity. Of particular interest in this regard was the absence of staining in the exocrine and endocrine pancreas.

Streptozotocin (STZ) in β-cells of the TGFα-βgal Transgenic Mice

STZ is a chemical inhibitor of O-GlcNAcase. Because pancreatic β-cells express high levels of O-GlcNAc transferase, they are particularly dependent on the reverse enzyme, the O-GlcNAcase. Results presented above suggest that when streptozotocin blocks this enzymatic activity, the proteasome subunit Rpt2/S4 is O-GlcNAcylated, leading to inhibition of proteasome function and subsequent accumulation of proteasome substrates that result in apoptosis in β-cells. Sp1 and p53 are proteasome substrates and these transcription factors both drive expression of the TGFα promoter. Hence, it is hypothesized that the TGFα promoter would be turned on by proteasome inhibition.

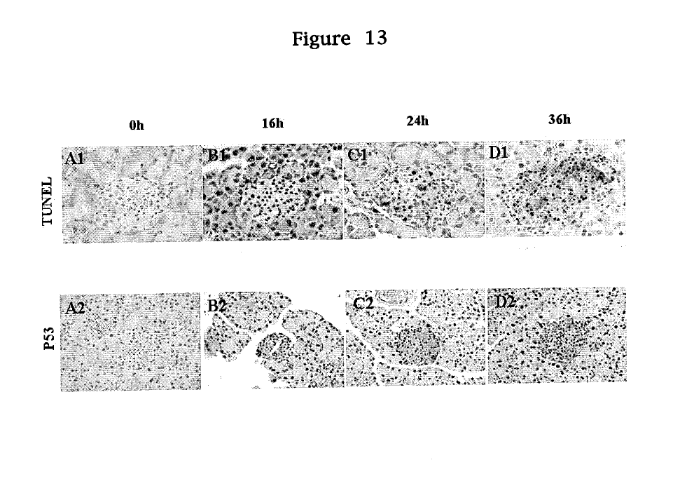

The effect of streptozotocin on p53 accumulation in the islets was determined using immunohistochemistical analysis of pancreatic slices from rats treated for various times with STZ. As shown in the upper portion of FIG. 13, streptozotocin treatment causes apoptosis as measured using the TUNEL assay. This assay has been described thoroughly (Liu et al., 2000). The TUNEL positivity peaks at about 22-24 hours after the administration of STZ. When the tissue was stained with p53 antibodies, it was noted that p53 accumulation preceded the apoptosis. At 16 hours, TUNEL positivity was only barely detectable, but p53 accumulation was well established. Of note is the reproducible observation that the exocrine pancreas does not show alterations in response to STZ. This corresponds with the observation the O-GlcNAc transferase is much more highly expressed in the endocrine pancreas. STZ also is toxic to the somatotropes in the pituitary where O-GlcNAc transferase is highly expressed. Thus, it is predicted there where O-GlcNAc transferase is abundant, STZ-sensitivity should be seen.

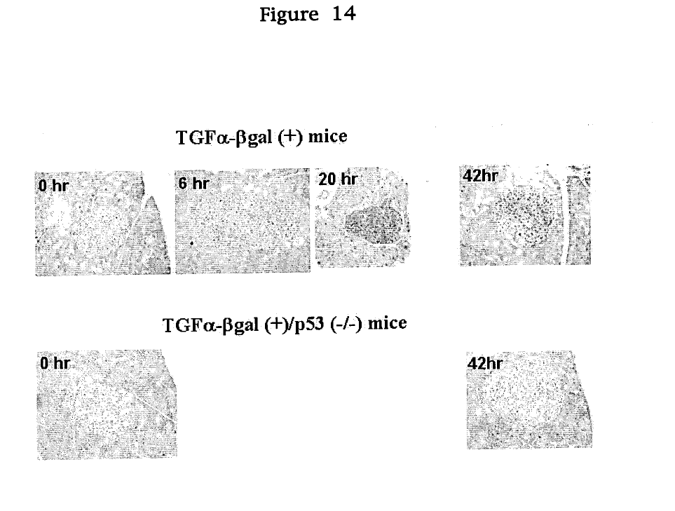

Because p53 accumulates in the islet in response to STZ, and TGFα transcription is driven by p53, it is reasonable to examine the TGFα reporter mice for TGFα promoter activation in the islets. Mice were injected with streptozotocin at the dose that cause β-cell apoptosis. The tissue sections were then examined for the appearance of reporter function, β-gal, as a function of time. FIG. 14 shows that β-gal activity appeared in the β-cells but not exocrine pancreas during a time course that paralleled the appearance of p53.

To determine if p53 was necessary for this response, the TGFα-β-gal reporter mice were bred with p53-knock-out mice. I n order to develop homozygous p53 knock-outs, two copies of the knocked out gene (homozygous) have to be present in addition to one copy of the TGFα reporter. Animals were screened using PCR analysis of tail DNA. The presence of the neo-cassette and the absence of wildtype p53 were assessed by PCR. The presence of the TGFα-βgal transgene was determined by PCR using one probe in the TGFα promoter and the other in the β-gal sequence. Thus, these animals were selected for three independently segregated genes. The probability of obtaining such an animal was 1 in 8. However, the p53 knock-out mice are susceptible to early onset cancer, so there is a negative selection for the homozygotes such that the overall probability of obtaining the three gene in one animal is more likely 1/20. A few of these animals have been obtained, and thus far, there was no TGFα reporter function in the islets in response to streptozotocin (FIG. 14). Thus, the preliminary results indicate two important points. First, TGFα reporter function is absent in resting β-cells, and can be stimulated by streptozotocin treatment. Second, TGFα reporter function appears to be p53-dependent in these cells. Hence, for pancreatic β-cells, the TGFα-βgal reporter behaves effectively as a p53 reporter when used in the islets.

STZ And The Brain of The TGFα-βgal Transgenic Mice

The hypothesis that underlies the present invention is that O-GlcNAcylation of proteasome blocks proteasomal function. This blockade of proteasomal function not only results in the characteristic aggregates of neurodegeneration but also accumulation of proapoptotic factors leading to the death of the affected neurons. Furthermore, cells with the highest levels of O-GlcNAc transferase expression should display the greatest sensitivity of O-GlcNAcase blockade since it is these cells that have the highest dependence on this enzyme to reverse the modification of proteins by O-GlcNAc transferase. If this hypothesis is correct, then chemical blockade of the O-GlcNAcase with streptozotocin should result in at least some of these features especially in those cells with the greatest O-GleNAc transferase expression.

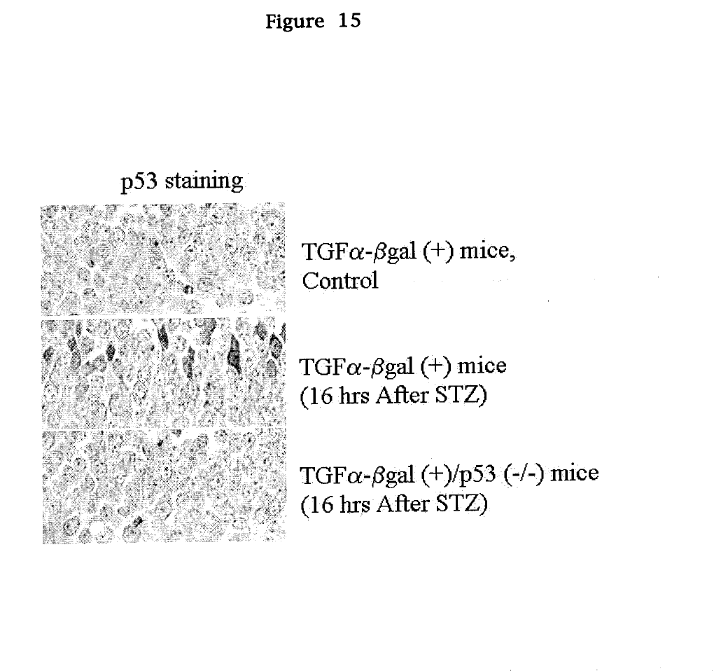

A number of research groups have developed techniques of injecting streptozotocin into the cerebral ventricles (Lannert & Hoyer, 1998; Weinstock et al., 2001; Sharma & Gupta, 2002). To determine if treatment with streptozotocin causes p53 accumulation in the brain, freshly prepared streptozotocin can be injected stereotactically into the cerebral ventricles, and the brain tissues can then be stained for the presence of p53. When the control TGFα-βgal mice were injected with vehicle alone, no p53 was detected 16 hours post injection. However, when these mice were injected with STZ, p53 was readily detectable by immunohistochemistry in the neurons of the hippocampus (FIG. 15) and Purkinje cells of the cerebellum (not shown). STZ was also injected into TGFα-βgal mice with a homozygous knock-out of p53. As expected, p53 did not accumulate in the hippocampal neurons nor Purkinje cells.

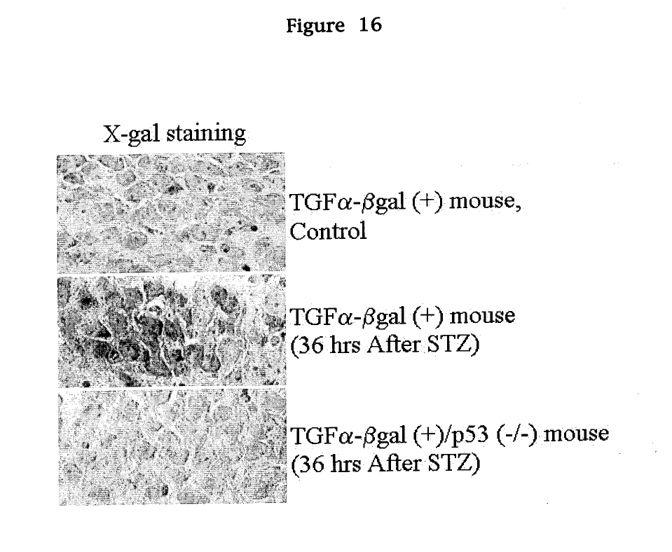

The brains slices from similar mice receiving icv injections 36 hours prior to sacrifice were examined for TGFα-βgal reporter function. FIG. 16 shows that the hippocampal neurons of the wildtype TGFα-βgal mice show reporter function and turn blue when exposed to X-gal. The p53 knock-out mouse displays no reporter function after icv streptozotocin injection.

### EXAMPLE 6

The Effects of Chemical Blockade of O-GlcNAc Catabolism In The Brain The above results show accumulation of p53 in the hippocampus and the activation of the TGFα-βgal reporter. The reporter function depends on p53, since p53 knock-out animals fail to demonstrate reporter activation. Since p53 accumulates because its proteasomal degradation is blocked, it is reasonable to assume that streptozotocin, by blocking the removal of O-GlcNAc by the O-GlcNAcase, causes proteasomal blockade due to the accumulation of O-GlcNAc on the Rpt2/S4 ATPase. Because hippocampal neurons contain more OGT that the surrounding tissue in the brain, this effect of streptozotocin would be more pronounced in these neurons than other brain regions intracerebroventricular

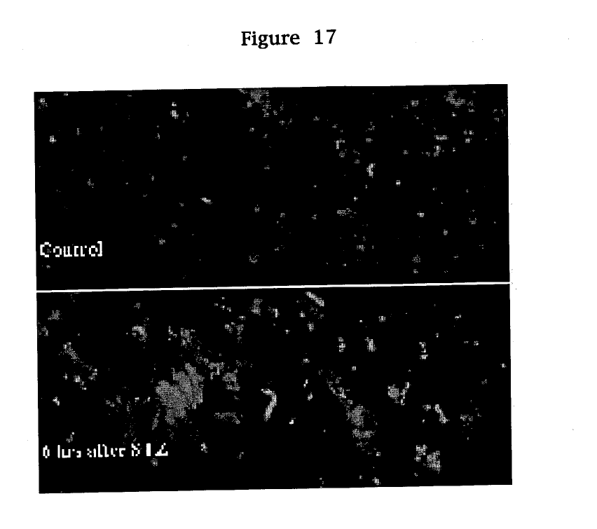

To determine if streptozotocin causes hippocampal neuron O-GlcNAc accumulation, the drug was delivered stereotactically to the cerebroventricles of a rat brain. The brain tissues were then stained for O-GlcNAc content using the O-GlcNAc-specific monoclonal antibody, RL2. Staining of rat tissue by this mouse antibody is easier because detection is not hampered by endogenous mouse IgG. Staining is blocked by preincubation of RL2 with GlcNAc, but not GalNAc or glucosamine (needs acetylation) or other sugars. The bound RL2 can be detected by fluorescent-tagged antimouse IgG antibody. FIG. 17 shows a preliminary experiment in which animals were injected with or without streptozotocin. At 6 hours post-injection, the animals were sacrificed and brain O-GlcNAc content was detected with RL2 immunostaining as previously described (Liu et al., 2000). Shown are hippocampal neurons (CA1 region) with a marked increase in the O-GlcNAc signal 6 hours after injection of streptozotocin. These results confirm that streptozotocin causes a marked accumulation of O-GlcNAc in the neurons shown to contain high OGT content.

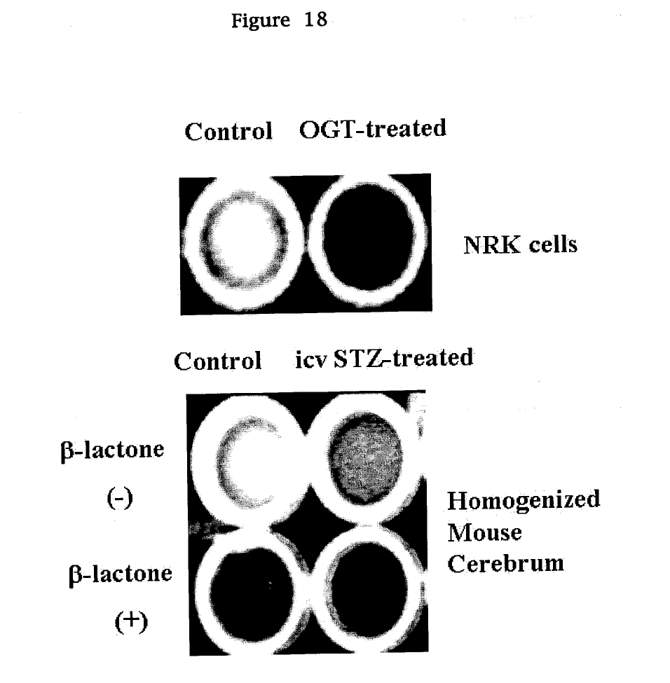

To determine whether STZ-injected animals display a n alteration in proteasomal function, the cleavage of the peptide LLVY was examined following streptozotocin treatment. Brain extract was prepared from animals injected with or without streptozotocin, and suc-LLVY-AMC was added to the extract. Using the 96-well plate assay and UV light at 365 nm to excite fluorescence, preliminary results demonstrated that there was a marked inhibition of the LLVY chymotryptic activity in the brains of animals treated with streptozotocin (FIG. 18). The fluorescence can be inhibited with a bona fide proteasome inhibitor, β-lactone. These results can b e refined in two ways. First, the use of the fluorometer will make these results more quantitative. Second, various regions of the brain can be dissected to determine if the inhibition of proteasomal function is region-specific.

Alternatively, the O-GlcNAc modification of the Rpt2/S4 ATPase subunit of the proteasome can be mesaured by western blot. Brains extract can be subjected to RIPA buffer which contains SDS, Triton X100 and deoxycholate to disrupt the proteasomes. Rpt2/S4 will be immunoprecipitated and western blotted with RL2 antibodies. Using this technique, it can be determined if streptozotocin treatment causes the predicted O-GlcNAcylation of this proteasomal subunit in vivo.

Proteasomal Blockade and Initiation of Neurodegenerative Cascade

For Alzheimer's disease, it is not clear what initiates the accumulation of Aβ to sufficient levels to aggravate the proteasomal defect. To initiate the proteasomal defect, the inventors will administer streptozotocin intracerebroventricularly on one occasion and follow the progression of brain pathology over the subsequent 1 or 2 months. Even though the effect of streptozotocin itself should subside with time (it has a short half-life), the proteasomal defect may be sufficient to initiate the accumulation of Aβ such that the positive feedback loop resulting in further inhibition of the proteasome continues. Thus, it is predicted that Aβ should continue to accumulate along with the apoptotic proteins that are normally cleared by the proteasome. The theory also would predict that proteasome function would continue to be inhibited. On the contrary, the accumulation of O-GlcNAc following streptozotocin should be a s short lived as the streptozotocin effect. That is, we should at first see an association of proteasome inhibition and O-GlcNAc accumulation but as Aβ accumulates to inhibit the proteasome, the requirement for O-GlcNAc abates. This idea would be true unless the O-GlcNAcase function is altered by the apoptotic process. Interestingly, it has been shown that O-GlcNAcase is cleaved by caspase-3, a key component of the apoptotic pathway. Hence, the initiation of the apoptotic process might destroy the very enzyme that would stop the process from proceeding, contributing further to the positive feedback loop of proteasomal inhibition (Bence et al., 2002).

The accumulation of O-GlcNAc, Aβ, tau, p53, p21, MDM2 and Bax can be followed in tissue sections in ‘replicate’ animals as a function of time following icv streptozotocin. In situ TUNEL assays (Liu et al., 2000) are also performed to determine at which point neuronal apoptosis occurs. The expectation is that streptozotocin will initiate the apoptotic cascade which will continue to self-perpetuate.

Similar experiments can be performed in the TGFα-βgal transgenic mice. A single dose of icv streptozotocin should initiate the cascade of events that lead to activation of the reporter via p53. The long-term activation of this reporter will support the idea that not only does p53 accumulate as a result of proteasome inhibition cascade, but p53 is functional. To determine whether p53 is integral for the perpetuation of the cascade, streptozotocin is injected into homozygous p53 knock-out mice and a similar time course of monitoring is done.

Behavioral Phenotyping of the Chemical and Transgenic Mouse Models

Behavioral consequences such as motor coordination, short term memory, anxiety, spatial orientation and learning ability can b e examined as described (Lalonde et al., 2002). After a 2-week adaptation period to the new surroundings and to handling by the experimenters, behavioral tests will be conducted in the morning and early afternoon for 21 days. The testing schedule included exploration of the T-maze (days 1-10), the open-field (days 1-3), the photocell activity chamber (days 4-6), and the elevated plus-maze (days 7-8), emergence from a small toy object (days 9-10), motor coordination on the stationary beam (day 11), the coat-hanger (day 12), and the rotorod (days 13-15), and spatial learning in the water maze (days 16-21). The tests will be performed with the apparatus and the result will be analyzed statistically as previously described.

### EXAMPLE 7

Genetic Blockade of Proteasomal Function

This example describes the development of transgenic mouse models in which proteasome function is blocked in the brain b y expressing a mutant form of a vital proteasome ATPase, a mutant ubiquitin or an O-GlcNAcase antisense in the brain. The pathology that results in the brain and the functional changes leading to behavioral alterations will be followed as described above.

The inventors propose to shut down proteasome function with temporal and tissue control. One of the six ATPase in the 19S cap of the proteasome is Sug1. It has been shown that addition of the ATPase mutant of Sug1 to nuclear extract inhibits proteasomal function. Although transient expression of mutant Sug1 in NRK cells using vaccinia virus does block proteasome function in vivo, implying that the mutant Sug1 is effective in vivo as a dominant negative, the phenotype of these cells cannot be studied once they are infected with the vaccinia virus. Furthermore, cell lines that non-conditionally express mutant Sug1 cannot be developed. Presumably, inactivation of proteasomes may have been incompatible with the growth or survival of cells in culture (congruent with the notion that proteasome inhibition causes apoptosis). Thus, if mutant Sug1 is to be expressed in vivo, it must be done conditionally so that transgene expression is not incompatible with life. In order to study the effect of proteasome blockade in the brain, tissue specificity is also required. The use of a transgenic model will provide relevance to the in vivo physiology and will obviate questions related to the genetic background of cell lines that, for instance, may be resistant to apoptosis because they have mutations in the apoptotic pathway.

The Tetracycline Inducible System

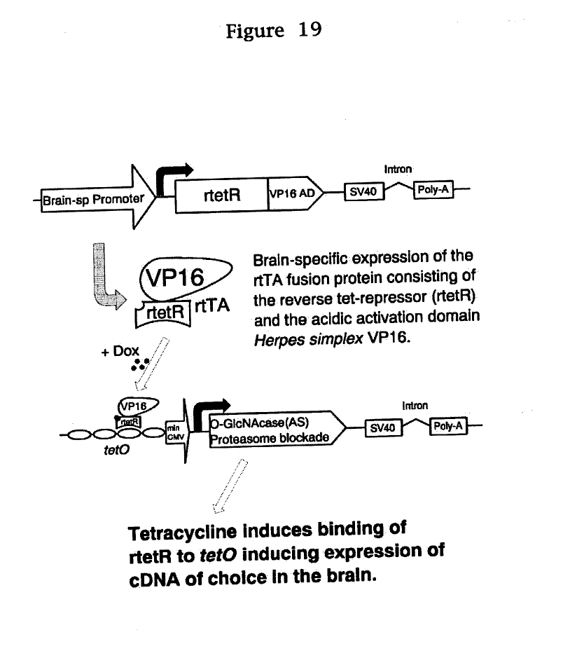

The tetracycline inducible system has been used to target conditional expression of transgenes to various organs. A schematic of this system is shown in FIG. 19 as it applies to brain. The VP16 activation domain in the tetracycline-dependent activator results in greater expression than is usually obtained from the tissue specific promoter. Since a dominant negative strategy requires that the level of expression of the dominant negative molecule significantly exceeds the level of the native molecule, this approach will be very advantageous for the proposed experiments.

The tetracycline system is therefore ideal for the proposed studies. This system can be used to test the hypothesis that any inhibitory perturbation to the proteasome, even temporarily, should be perpetuated because of the positive feedback loop by which Aβ accumulation continues to inhibit proteasomes. Since the tet-on system can be reversibly activated by the administration or removal of doxycycline, the temporal requirements of proteasome inhibition can be tested.

A tet-activator mouse that drives the tet-activator, rtTA, expression in the relevant areas of the brain has already been developed by Eric Kandel. The CaMKIIα promoter has been used to drive brain-specific expression of rtTA. Other neuron specific promoters have also been applied to this system. The PDAPP mouse is a model of Alzheimer's disease in which APP and its mutants has been overexpressed in the brain using the PDGF-β chain promoter (183). These mice recapitulate many of the feature of Alzheimer's including the formation of Aβ plaques in the areas of the brain such as hippocampus and have been used, for example, in several publications to investigate Aβ load reduction by immunization.

The inventors have obtained the same PDGF-β gene promoter used to generate this model and constructed a transgene which will direct the expression of rtTA to the same regions of the brain as the APP in the PDAPP mice. The construct, whose schematic is shown in FIG. 19, has already been microinjected in the UAB transgenic mouse facility. Gene positive founders will be bred with βgal reporter mice obtained from Julie Segre. These mice, first developed by Hennighausen and co-workers have a TetRE-βgal transgene driven by the tet-operon. The resulting bitransgenic mice will be tested to determine if the expression of the βgal reporter is tetracycline-dependent. The tissue will be stained as described above. Brain sections and sections of other tissues will be studied to determine if expression of the reporter is site-specific.

Transgenic With Mutant Sug1

The inventors have established a transgenic model with mutant Sug1 (TetRE-mSug1). The mSug1 construct contains the Xpress and HIS tag epitopes for which monoclonal antibodies are available. Mutant Sug1 RNA has been detected by in situ hybridization in the skin and pituitary of bitransgenic mice treated with doxycycline. Conditional expression of mSug1 can also b e examined by immunostaining for the epitope tags. As mentioned, the ATPase mutation in Sug1 blocks proteasome function in vitro and in transfected cells. Therefore, proteasome function is expected to be conditionally blocked in the brain regions of the bitransgenic mice. Proteasome blockade will be determined by measuring LLVY cleavage and Sp1 cleavage. Once confirmed, this mouse model can be used to test the idea that inhibition of proteasomes in the brain results in the accumulation of aggregates and the apoptotic death of nerve cells. The aggregates will be detected by immunocytochemistry, the apoptotic death will be followed by immune detection of p53 and its targets and by in situ TUNEL assay as described above. Tri-transgenic mice (PDGF-rtTA/TetRE-mSug1+TGFα-βgal) can also be generated to determine if proteasome blockade turns on the TGFα-βgal reporter.

It is expected that once proteasome inhibition is initiated, it continues to cascade as a result of the accumulation of aggregates. The reversibility of the tetracycline induction of the transgene is crucial for this experiment. Animals are fed doxycycline for various times from 6 hours to continuously. After these timed exposures to doxycycline, the animals will be followed for one or two months to determine if the pathological and behavioral consequences of proteasome blockade continue to accrue. Such a finding would support the Aβ hypothesis which basically states that once aggregates begin to accumulate, they inhibit the proteasome and result in the continued spiral that leads to neuronal loss by apoptosis and the characteristic pathological aggregates. These results would provide a baseline to evaluate the consequences of proteasome blockade in the brain for comparison with the streptozotocin model and the antisense O-GlcNAcase model described below.

Transgenic With Mutant Ubiquitin

Atlernative means of proteasomal inhibition can be generated. It has been shown that proteasomes can be inhibited by polyubiquitin conjugates that contain the mutant form of ubiquitin resulting from the molecular misreading of the ubiquitin mRNA. These conjugates are found in Alzheimer plaques and inhibit proteasomal function on cells in vivo. The cDNA encoding the misread form of ubiquitin, UBB⁺¹, can be placed downstream of the tetresponse element to create the transgene TetRE-UBB⁺¹. Bitransgenic mice will be used to express this abnormal ubiquitin in the brain regions of mice to determine if the Alzheimer's cascade can be initiated by this means of proteasomal inhibition. The experiments will be as outlined for the tagged mSug1.

Transgenic With O-GlcNAcase Antisense

To develop the idea that the O-GlcNAcase gene is a functional candidate for the pathogenesis of Alzheimer's disease, the expression of this gene can be blocked to see if the consequences of this blockade are similar to the proteasome blockade described above. STZ, a chemical inhibitor of the O-GlcNAcase gene does cause a neurodegenerative disorder when injected icv. The data strongly suggests that O-GlcNAcase blockade induced non-competitively b y streptozotocin results in the failure to cycle O-GlcNAc off the ATPase subunit Rpt2/S4 of the 19S cap of the proteasome. This glycosylated subunit is proposed to inhibit the proteasome degradation of aggregate and apoptotic proteins leading to the characteristic neurodegeneration. However, it remains possible that streptozotocin inhibits proteasomal function by another mechanism besides affecting to the O-GlcNAcase.

To directly test the role of O-GlcNAcase, four gene-positive antisense O-GlcNAcase transgenic mice have been developed. To create this transgene, the mouse O-GlcNAcase cDNA including the entire 5′-UTR was cloned by 5′-RACE. The entire 5′-UTR and coding sequence of the mouse O-GlcNAcase was subcloned in reverse (and forward) orientation downstream of the TetRE promoter. When this promoter is driven by the activator (rtTA) in bitransgenic mice, a n antisense RNA will be conditionally expressed under doxycycline induction in the brain in a distribution dictated by the PDGF-β promoter. The methods for quantitating antisense expression is published in full detail (Liu et al., 2000). The consequences of antisense expression are studied as described above.

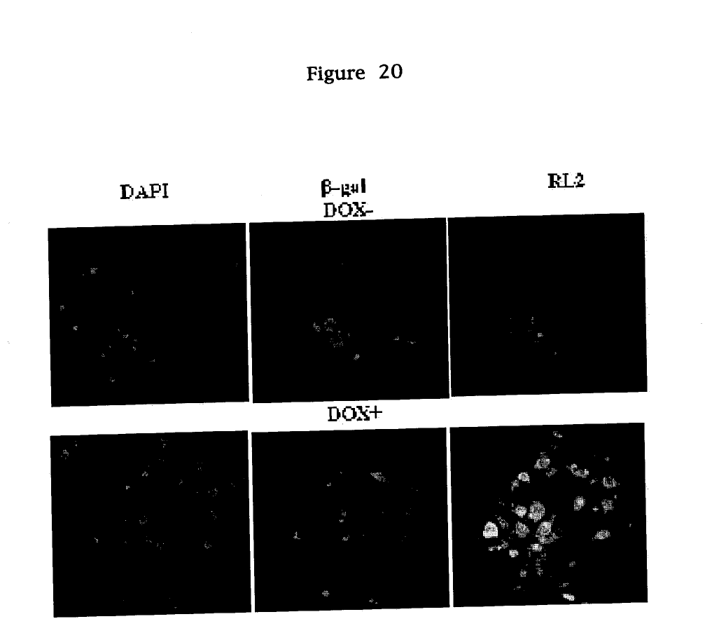

To develop a proof of principle that the antisense O-GlcNAcase will block expression of the enzyme, 3T3 mouse cells were transfected with TetRE-O-GlcNAcase(AS) (the actual transgene), CMV-rtTA and CMVO-gal. The transfected cells were identified by β-gal immunostaining and the O-GlcNAc content in the cells was determined with RL2 antibody immunofluorescence. The cells were treated with or without doxycyline to conditionally induce antisense expression. As shown in FIG. 20, induction of antisense expression results in a marked increase in RL2 staining as compared to the same cell pool that did not receive doxycyline. This result indicates the accumulation of O-GlcNAc modification both in the cytoplasm and nucleus of the transfected cells, thus demonstrating the effectiveness of the antisense construct used to generate the transgenic mice.

To establish that the antisense construct is conditionally expressed, the TetRE-O-GlcNAcase(AS) mice will be crossed with the K14-rtTA mice. The K14 mice are well established conditional activators for skin expression. Following a time course of doxycycline, skin biopsy samples will be obtained from the bitransgenic mice. The samples will be examined by immunostaining, northern blotting and/or in situ hybridization with a sense riboprobe (Liu et al., 2000) to determine if there is a time dependent accumulation of the antisense RNA molecule and its immediate consequence, O-GlcNAc accumulation.

The experiments with streptozotocin and proteasome blocking transgenes are repeated in the O-GlcNAcase(AS) mice to determine if the same consequences of proteasomal blockade occur with O-GlcNAcase blockade. With the transgenes, the promoter (PDGF-β) will be kept constant so that the same regions of the brain should display either proteasome blockade or O-GlcNAcase blockade. If the resultant Aβ cascade, pathology and behavioral changes occur, it will be a strong correlate between the biochemistry of modification of the proteasomal ATPase by O-GlcNAc and the pathological consequences of its alteration in the brain.

The following references were cited herein:

Bence et al., Science 292:1552-1555 (2002).

Bertram et al., Science 290:2302-2303 (2000).

Ertekin-Taner et al., Science 290:2303-2304 (2000).

Gao et al., J. Biol. Chem. 276:9838-9845 (2001).

Konrad et al., Biochem. Biophys. Res. Commun. 267:26-32 (2000).

Konrad et al., Biochem. J. 356:31-41 (2001a).

Konrad et al., Biochem. Biophys. Res. Commun. 288:1136-1140 (2001b).

Konrad et al., Biochem. Biophys. Res. Commun. 293:207-212 (2002).

Lalonde et al., Brain Res. 956:36-44 (2002).

Lannert & Hoyer, Behav. Neurosci. 112:1199-1208 (1998).

Liu et al., Proc. Natl. Acad. Sci. U.S.A. 97:2820-2825 (2000).

Myers et al., Science 290:2304-2305 (2000).

Sharma & Gupta, ChrLife Sci 71:2489-2498 (2002).

Weinstock et al., Ann N Y Acad Sci 939:148-161 (2001).

Yang et al., Cell 110:69-80 (2002).

Any patents or publications mentioned in this specification are indicative of the levels of those skilled in the art to which the invention pertains. These patents and publications are herein incorporated by reference to the same extent as if each individual publication was specifically and individually indicated to be incorporated by reference.

What is claimed is:

1. A method of inhibiting neuro-endocrine cell death, comprising the step of:
contacting said neuro-endocrine cell with an inhibitor that inhibits the enzymatic activity of O-linked N-acetylglucosamine transferase, wherein inhibition of O-linked protein glycosylation resulting from inhibition of said enzyme leads to inhibition of neuro-endocrine cell death.

2. The method of claim 1, wherein said inhibitor is a structural analog of N-acetylglucosamine.

3. The method of claim 2, wherein said analog is (Z)-1-[N-(3-Ammoniopropyl)-N-(n-propyl)amino]diazen-ium-1,2-diolate or a derivative thereof.

4. The method of claim 1, wherein said inhibitor is a uracil-like drug.

5. The method of claim 4, wherein said uracil-like drug is alloxan.

6. The method of claim 1, wherein said neuro-endocrine cell is selected from the group consisting of pancreatic β-cell, cells of the central nervous system including the brain and spinal cord and other hormone-secreting cells including the pituitary gland.

7. A method of treating or inhibiting the onset of diabetes mellitis in an individual, said method comprises the step of:
administering to said individual a pharmacological dose of a compound that inhibits the enzymatic activity of O-linked N-acetylglucosamine transferase in a tissue or cell of said individual, wherein inhibition of O-linked protein glycosylation resulted from inhibition of said enzyme would lead to prevention or treatment for diabetes mellitis.

8. The method of claim 7, wherein said compound is a structural analog of N-acetylglucosamine.

9. The method of claim 8, wherein said analog is (Z)-l[N-(3-Ammoniopropyl)-N-(n-propyl)amino]diazen-ium-1,2-diolate or a derivative thereof.

10. The method of claim 7, wherein said inhibitor is a uracil-like drug.

11. The method of claim 10, wherein said uracil-like drug is alloxan.

12. The method of claim 7, wherein said tissue or cell is pancreatic beta-cells.

13. A method of treating or inhibiting late onset of Alzheimer's disease in an individual, comprising the step of:
administering to said individual a pharmacologically effective dose of a compound that inhibits the enzymatic activity of O-linked N-acetylglucosamine transferase in the brain cells of said individual, wherein inhibition of O-linked protein glycosylation resulting from inhibition of said enzyme leads to prevention or treatment for late onset of Alzheimer's disease.

14. The method of claim 10, wherein said compound is a structural analog of N-acetylglucosamine.

15. The method of claim 13, wherein said analog is (Z)1-[N-(3-Ammoniopropyl)-N-(n-propyl)amino]diazen-ium-1,2-diolate or a derivative thereof.

16. The method of claim 13, wherein said inhibitor is a uracil-like drug.

17. The method of claim 16, wherein said uracil-like drug is alloxan.
